(19) United States
(12) Patent Application Publication
(10) Pub. No.: US 2005/0043685 A1
(43) Pub. Date: Feb. 24, 2005

(54) Device for fixing a tube within a hole in a body tissue layer

(76) Inventor: Delia Schinkel-Fleitmann, Gottingen (DE)

Correspondence Address: Scott A. Horstemeyer;THOMAS, KAYDEN, HORSTEMEYER & RISLEY, L.L.P., Suite 1750, 100 Galleria Parkway, Atlanta, GA, 30339, US

(21) Appl. No.: 10/953,254

(22) Filed: Sep. 29, 2004

Related U.S. Application Data
(63) Continuation of application No. PCT/EP03/03164, filed on Mar. 28, 2003.

(30) Foreign Application Priority Data
Mar. 30, 2002 (DE) 102 14 414.1-35

(51) Int. Cl. A61B 17/04

(52) U.S. Cl. 604/174

(57) ABSTRACT

A device for fixing a tube having a smooth wall within a hole in a body tissue layer having an exterior and an interior comprises a L-shaped retaining element having two limbs, and a fixing mechanism for fixing one of said two limbs of said retaining element to the wall of the tube, after the other of said two limbs of the retaining element has been inserted from the exterior of the body tissue layer through the hole to the interior of the body tissue layer and placed flat on the body tissue layer at the interior of the body tissue layer.

### CROSS REFERENCE TO RELATED APPLICATIONS

This application is a continuation in part of International Patent Application PCT/EP03/03164 filed Mar. 27, 2003 and claiming priority to co-pending German Patent Application No. 102 14 414.1 filed Mar. 30, 2002 both of which are entitled “Vorrichtung zur Fixierung eines Trokars, eines Schlauchs oder Katheters”.

### FIELD OF THE INVENTION

The present invention generally relates to fixing a tube within a hole in a body tissue layer. More particular the present invention relates to a device for fixing a trocar tube, a drain tube or a catheter tube having a smooth wall within a hole in a body tissue layer having an exterior and an interior, especially in a skin or body wall.

### BACKGROUND OF THE INVENTION

In endoscope operations, such as laparoscopy, metal or plastic tubes called trocars are inserted through the skin and the adjacent body wall into the interior of the body to provide a working channel. Through the trocar tube, the surgeon may insert an optical or surgical instrument, such as an endoscope or a pair of scissors, into the body cavity. By changing the instruments, such as changing pliers for scissors, and by moving the instruments to-and-fro, a considerable mechanical load is placed on the trocar tube. To at least partially bear this load, an assistant of the surgeon normally firmly holds the used trocar tubes. However, it happens frequently that a trocar tube slips out of the hole in the skin or gets into this hole too deeply, because, as a rule, more than two trocars are used in one operation. This results in delays, makes the operation more difficult, and may lead to involuntary injuries and complications.

Known devices for fixing trocar tubes comprise a thread at the outside of the trocar tube, by which the respective trocar tube is screwed into the tissue layer delimiting the hole in the body tissue layer.

Further, screw-like additional trocars are known in different sizes, a trocar tube enclosing the instruments and having a smooth wall being placed into the additional trocar and then being inserted into the hole in the tissue layer. Here, fixation of the trocar tube with regard to the additional trocar is possible in various positions. However, it has to be paid for this in that, besides additionally traumatizing the skin over its entire depth by screwing-in the additional trocar, a longer cut is required for a bigger hole which in addition to the trocar tube also has to accommodate the additional trocar.

Independently of the fact whether trocar tubes or additional trocars are provided with outside threads, they are only available at comparatively high cost.

Non-traumatic devices for fixing trocar tubes, i.e. fixing devices which do not cause injuries are not available up to now. A further area of application of the invention is the fixation of drain tubes and catheter tubes, particularly long time drains and long time catheters. Here, it is required up to now to sew the drain tube or catheter tube to the skin of a patient.

Thus, there is a need for a device for minimum-traumatically fixing a tube within a hole in a body tissue layer, which can be used in combination with existing smooth wall trocar tubes, drain tubes and catheter tubes, and which can be provided at low-cost.

### SUMMARY OF THE INVENTION

The present invention relates to a device for fixing a tube having a smooth wall within a hole in a body tissue layer having an exterior and an interior, the device comprising a L-shaped retaining element having two limbs, and a fixing mechanism for fixing one of said two limbs of said retaining element to the wall of the tube, after the other of said two limbs of the retaining element has been inserted from the exterior of the body tissue layer through the hole to the interior of the body tissue layer and placed flat on the body tissue layer at the interior of the body tissue layer.

Further, the invention relates to a device for fixing a tube having a smooth wall within a hole in a body tissue layer having an exterior and an interior, the device comprising a L-shaped retaining element molded of plastic and having two limbs, and a fixing mechanism for fixing one of said two limbs of said retaining element to the wall of the tube, after the other of said two limbs of the retaining element has been inserted from the exterior of the body tissue layer through the hole to the interior of the body tissue layer and placed flat on the body tissue layer at the interior of the body tissue layer; said one limb comprising a channel for partially receiving the tube, a radius of curvature of said channel corresponding to an outer radius of the tube; said other limb having a flat arc-shaped cross-section, a radius of curvature of the cross-section at the lower side of said one limb facing away from said one limb corresponding to an outer radius of the tube, and said other limb comprising a contour at its upper side facing towards said one limb, which avoids the body tissue layer from slipping-off from said other limb; and the fixing mechanism comprising a hook protruding from said one limb, and an elastically extendible strap retainer having an end which is anchored to said one limb of the retaining element and a remainder which can be slung about the tube in said channel and then be hooked on said hook.

One aspect of the new device is the L-shaped retaining element. This retaining element has two limbs. With a fixed tube, one of these limbs runs in parallel to the tube and is fixed to the tube, while the other is placed flat on the interior of the body tissue layer. The body tissue layer generally is the outermost tissue layer of the skin (cutis). The other limb of the retaining element abutting against the tissue layer securely hinders involuntarily pulling the tube out of the hole in the tissue layer. Further pushing the tube into the hole, which is also unwanted, is hindered by the fixing mechanism for fixing one limb of the retaining element to the tube. The one limb or the fixing mechanism may also comprise special stops for abutting against the exterior of the body tissue layer.

For installing the new device, the tube may be inserted into the hole first, and then the other limb of the L-shaped retaining element may be inserted into the hole in parallel to the tube and afterwards, when it is already at the interior of the tissue layer, be folded on the tissue layer so that the one limb of the retaining element is aligned in parallel to the tube. Next, the tube and the one limb of the retaining element aligned in parallel to the tube are fixed to each other so that the tube is fixed with regard to the hole. An additional extension of the hole in the body tissue layer is not required for using the new device to any significant extent. There is also no significant additional stress on the body tissue layer by the retaining element. At the same time, the device may be provided at low cost and it may be used with already existing smooth wall tubes like, for example, trocar tubes. An adaptation of the device to the actually used tube at least to a certain extent is sensible. A precise matching to the respective outer diameter of the tube, however, is not required.

If the other limb of the retaining element has a flat arc-shaped cross section, and if a radius of curvature of the cross section at the lower side of the other limb facing away from the inside of the tissue layer corresponds to the outer radius of the tube, the other limb may be particularly easily brought to the interior of the tissue layer next to the tube already inserted into the hole. Upon being pushed through the hole, the other limb increases the total outer diameter of the tube and the fixing device in the hole to an only insignificant extent. Particularly, if the one limb of the retaining element has a flat profile with a low wall thickness, it has to be made sure that there is no danger of breaking at the interior of the body tissue layer. The arc-shape of the cross section of the limb, however, results into a high dimensional stability, even if the retaining element is made of plastic.

If the other limb of the retaining element is contoured at its upper side facing the interior of the body tissue layer, which stops the tissue layer from slipping off, the retention of the tube in the hole can be ensured to an even higher extent.

The one limb of the retaining element preferably comprises a channel for partially receiving the tube and abutting against the tube. By means of this, the parallel arrangement of the one limb and the tube is supported by form fit. This ideally applies, if the surface radius of curvature of the channel in the one limb at least essentially corresponds to the outer radius of the tube.

For fixing the one limb of the retaining element to the tube, one end of a strap retainer can be anchored at the one limb, which strap retainer may be slung around the tube and then be fixed to the one limb under elastic tension. Thus, the tube is held in its position with regard to the retaining element by the effect of static friction between its surface on the one hand, and the one limb and the strap retainer, respectively, on the other hand. The surface of the channel in which the tube abuts against the one limb may be coated with a suitable coating to have a particularly high static friction with regard to the tube.

The retainer strap may be an elastically extendible perforated tape which may be hooked on a hook protruding from the one limb after it has been slung around the tube. Particularly, a lateral opening may be provided in the one limb, the hook protruding into the path of the strap retainer slung around the tube and inserted through the opening. The hooked-on strap retainer may be removed from the hook by means of pulling it in the opposite direction to free the strap retainer and thus finally the tube for pulling it out of the hole, or for repositioning it with regard to the hole.

According to a particularly preferred embodiment of the invention, the retaining element with both limbs is constituted by a dimensionally stable one piece body molded of plastic. Generally, however, the retaining element may also be made of other materials, like stainless steel, for example.

The new device may also have two L-shaped retaining elements which are facing each other across the tube in fixing the tube. In this way, an even more secure anchoring of the tube within the tissue layer is achieved. Both L-shaped retaining elements are preferably molded bodies of plastic, which each form both limbs of one retaining element.

In a particular embodiment of the new device having two L-shaped retaining elements, the two molded bodies of plastic are identical, and they are connected to each other by means of the already described strap retainer.

In another particular embodiment of the new device having two L-shaped retaining elements, both molded bodies of plastic are attached to each other by a film hinge. Thus, they are molded of plastic in a common mold. The film hinge provides a swivel axis which runs approximately in parallel to the two one limbs and thus to the direction of main extension of the tube. On the side of the tube facing away from the film hinge a latching connection is provided between the two one limbs of the two molded bodies attached to each other. This latching connection latches-in around the tube upon compressing the molded bodies, and bears the pressure force of the molded bodies on the tube; which pressure force is required for applying the static friction fixing the one limbs of the molded bodies to the tube. This embodiment of the invention is particularly well suited for quickly fixing drain tubes, and also provides for the possibility of quickly exchanging the respective drain tube.

Other features and advantages of the present invention will become apparent to one with skill in the art upon examination of the following drawings and the detailed description. It is intended that all such additional features and advantages be included herein within the scope of the present invention, as defined by the claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 is a perspective view of a first embodiment example of a new device, showing it separately from one side.

FIG. 2 is a side view of the first embodiment of the new device of FIG. 1 in its use with a fixed trocar tube, the direction of view being opposite to that one of FIG. 1.

FIG. 3 is a perspective view separately showing a second embodiment example of the new device in an open position.

FIG. 4 is a view from below showing the second embodiment of the new device in the same position as in FIG. 3.

FIG. 5 is a view from below showing the second embodiment of the new device in a closed position; and

FIG. 6 is a perspective side view showing the second embodiment in the position according to FIG. 5 with a fixed drain tube.

### DETAILED DESCRIPTION

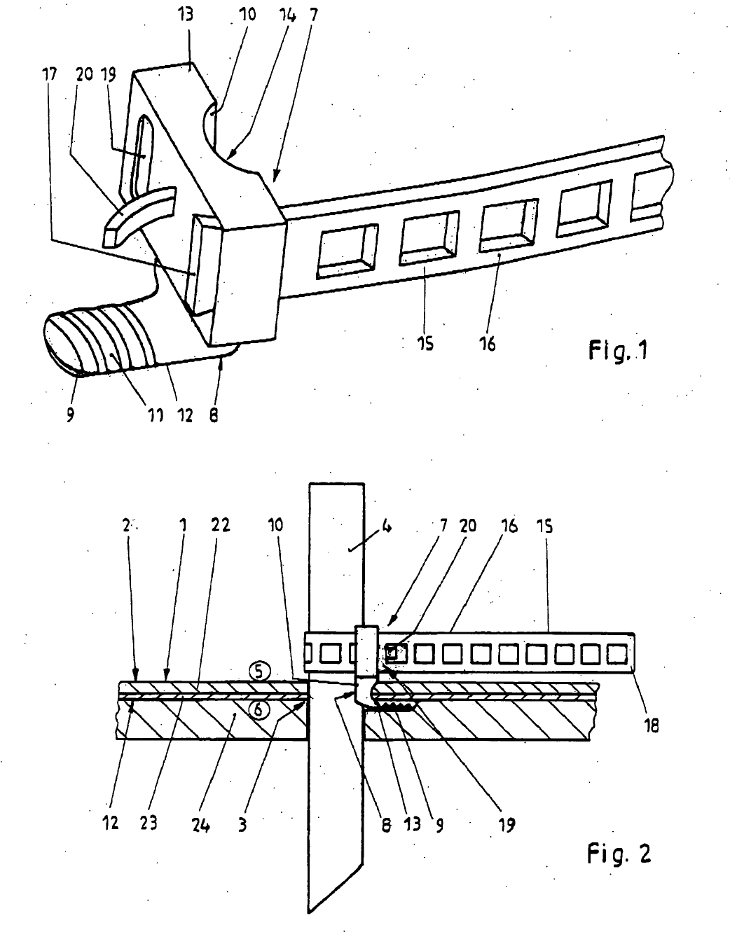

Referring now in greater detail to the drawings, FIGS. 1 and 2 illustrate a device 7 which is intended for fixing a tube, such as a trocar 4 tube shown in FIG. 2, with regard to a body tissue layer 1. The body tissue layer typically is a skin 2. The trocar tube has a smooth wall. A hole 3 is provided in the tissue layer 1, i.e. cut-in with a scalpel, through which the trocar tube 4 extends from an exterior 5 to an interior 6 of the body tissue layer 1. The device 7 includes an L-shaped retaining element 8 having two limbs 9 and 10. In the use of the device 7 according to FIG. 2, the limb 9 is arranged at the interior 6 of the tissue layer 1, and it is placed flat on the tissue layer 1 there. Particularly, it is arranged at the interior 6 of a cutis 12 consisting of an epidermis 22 and a corium 23 of the skin 2, i.e. it is still above a subcutis 24. An upper side of the limb 9 may be provided with a contour 11 as depicted in the figures to stop the tissue layer 1 from involuntarily slipping off the limb 9. The limb 9 is constituted by a one piece body 13 molded of plastic which also forms the limb 10. In the area of the limb 9 and in the transition region between the limbs 9 and 10 the molded body 12 is of flat arc-shape. The radius of curvature of the arc being matched to the outer diameter of the trocar tube 4, which enables inserting the limb 9 close to the trocar through the hole 3 to the interior 6 of the body tissue layer 1. In the area of the limb 10 the molded body 13 has a channel 14 which partially receives the trocar tube 4 and which causes an alignment of the trocar tube 4 in parallel to the limb 10. A strap retainer is provided for fixing the limb 10 to the trocar 4, which is an elastically extendible trellis tape or perforated tape 16. One end 17 of the strap retainer 15 is anchored to the molded body 13. The remainder of the strap retainer 15 is slung around the trocar tube 4. Then, the free end 18 of the strap retainer 15 is pulled through an opening 19 in the molded body 13 and tightened. The tightened strap retainer 15 is fixed on a hook 20 protruding from the molded body 13. The trocar tube 4 is thus fixed to the limb 10 of the retaining element 8 via its outer surface by means of static friction towards the strap retainer 15 and the molded body 13. This results into the desired fixation of the trocar tube 4 with regard to the hole 3 or the tissue layer 1, because the retaining element 8 abuts on the tissue layer 1 via the limb 9 in the one direction and via the further parts of the device 1 extending from the trocar tube 4 at the exterior 5 in the other direction. Releasing the trocar tube 4 is achieved in that the strap retainer 15 is tensioned to such an extent, that the hook 10 gets out of the strap retainer 15 again. Then, the strap retainer 15 can be pushed back through the opening 19. Next, either the trocar 4 or the device 7 can be removed out of the hole 3 first. The new device can be provided at low cost and is perfectly suited for use with existing smooth wall trocar tubes 4 and other tubes. Its use is associated with no significantly higher tissue invasion than anyway necessary for inserting the tube. There is also no additional traumatizing of the tissue worth to be mentioned. Instead, it is important that by means of stopping the trocar tube 4 from slipping out of the hole 3 a higher handling security, and thus finally a better operation result, is achieved within shorter time. Complications by involuntary injuries as a result of uncontrolled axial movements of the trocar tube or of instruments arranged in the trocar tube are definitively avoided by means of the new device.

Using the new device for fixing drain tubes and long term catheters, in which two molded bodies 13 facing each other may be used in connection with one strap retainer 15, means a quick fixation without sewing the tube to the skin of the patient. This saves time and money.

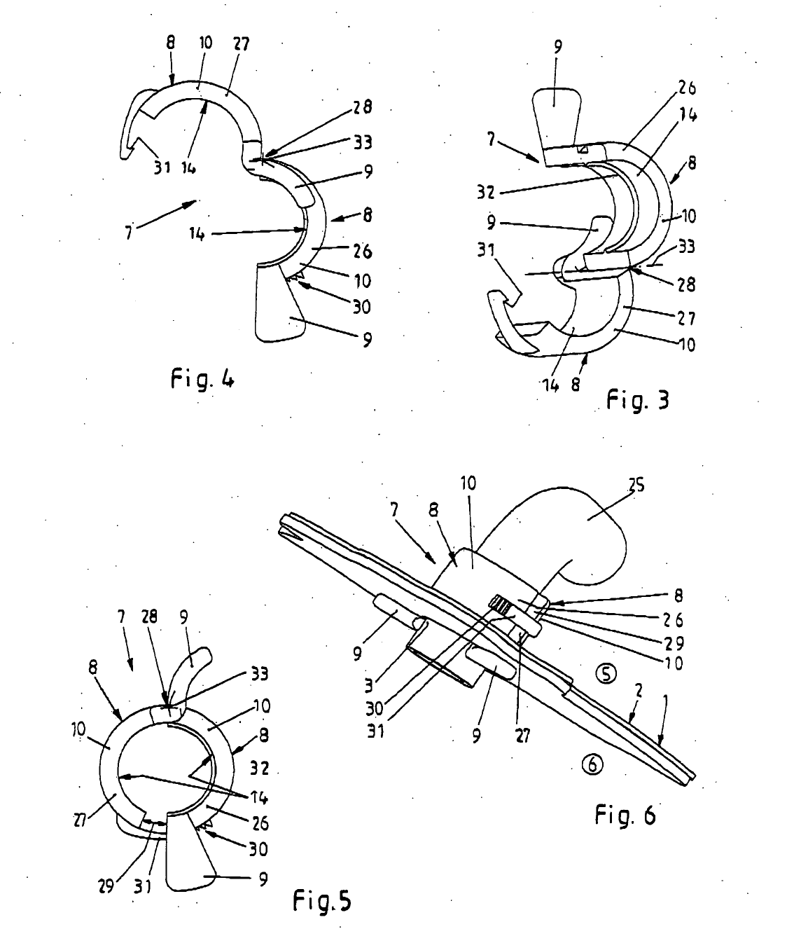

The second embodiment example of the new device shown in FIGS. 3 to 6 provides for a further possibility especially for fixing tubes of drains and long term catheters. One section of a drain tube 25 is depicted in FIG. 6. The device according to FIGS. 3 to 6 consists of two differently shaped bodies 26 and 27 molded of plastic which are attached to each other via a film hinge 28, and which each form one L-shaped retaining element 8. I.e. each molded body 26 and 27 comprises one limb 9 which is to be arranged at the interior 6 of the tissue layer 6, and one limb 10 which extends along the fixed drain tube 25. The channels 14 in the limbs 10 are formed in such a way, that they together nearly completely surround the drain tube 25. A small gap 29 remains on the side of the drain tube 25 opposite of the film hinge 28, to be able to compress the molded bodies 26 and 27 around the drain tube 25 in the position of the device 7 according to FIGS. 5 and 6 for providing a pre-stress on the drain tube 25. In this position the two molded bodies 26 and 27 are held by a latching connection 30, 31 consisting of saw teeth-shaped latching elements 30 at the molded body 26 and a hook 31 engaging the latching elements 30 and elastically supported at the molded body 27. A static friction is built up by the force applied on the drain tube 25 by the interlocked molded bodies 26 and 27, which is further increased by an edge 32 of the inner circumference in the channel 14 of the molded body 26. For fixing a drain tube 25, the limb 9 of the molded body 26 of the opened device 7 is inserted into the hole 3 within the tissue layer 1 next to the drain tube 25. Then, the other limb 10 of the molded body 26 is placed on the tube. In this step, the limb 9 of the molded body 27 is also inserted through the hole 3 under the tissue layer 1 surrounding the tube. The position of the device 7 reached in this way, in which the limb 9 of the molded body 27 is positioned below the limb 10 of the molded body 26, is particularly visible in FIG. 4. Next, the two molded bodies 26 and 27 are swivelled together about a swivel axis 33 defined by the film hinge 28 and running in parallel to the two limbs 10, until the hook 31 engages the latching elements 30 and until a sufficient pre-stress is placed upon the drain tube 25 to hold it in the device 7 by static friction. By raising the hook 31 from the latching elements 30 the drain tube is released, and the device 7 can then be removed out of the hole 3.

Many variations and modifications may be made to the preferred embodiments of the invention without departing substantially from the spirit and principles of the invention. All such modifications and variations are intended to be included herein within the scope of the present invention, as defined by the following claims.

1. A device for fixing a tube having a smooth wall within a hole in a body tissue layer having an exterior and an interior, the device comprising a L-shaped retaining element having two limbs, and a fixing mechanism for fixing one of said two limbs of said retaining element to the wall of the tube, after the other of said two limbs of the retaining element has been inserted from the exterior of the body tissue layer through the hole to the interior of the body tissue layer and placed flat on the body tissue layer at the interior of the body tissue layer.

2. The device of claim 1, wherein the tube is selected from a group consisting of trocar tubes, drain tubes and catheter tubes.

3. The device of claim 1, wherein said other limb has a flat arc-shaped cross-section, a radius of curvature of the cross-section at the lower side of said one limb facing away from said one limb corresponding to an outer radius of the tube.

4. The device of claim 1, wherein said other limb comprises a contour at its upper side facing towards said one limb, which avoids the body tissue layer from slipping-off from said other limb.

5. The device of claim 1, wherein said one limb comprises a channel for partially receiving the tube.

6. The device of claim 5, wherein a radius of curvature of said channel corresponds to an outer radius of the tube.

7. The device of claim 5, wherein the fixing mechanism comprises a strap retainer having an end which is anchored to said one limb of the retaining element and a remainder which can be slung about said channel and then also be fixed to said one limb.

8. The device of claim 7, wherein the fixing mechanism comprises a hook protruding from said one limb, and wherein said strap retainer comprises an elastically extendible perforated tape which can be hooked on said hook.

9. The device of claim 8, wherein a lateral opening is provided in said one limb, said hook protruding from said one limb into a path of said perforated tape slung about said channel and pulled through said opening.

10. The device of claim 1, wherein both of said limbs of said L-shaped retaining element are constituted by a one piece body molded of plastic.

11. A device for fixing a tube having a smooth wall within a hole in a body tissue layer having an exterior and an interior, the device comprising
a L-shaped retaining element molded of plastic and having two limbs, and
a fixing mechanism for fixing one of said two limbs of said retaining element to the wall of the tube, after the other of said two limbs of the retaining element has been inserted from the exterior of the body tissue layer through the hole to the interior of the body tissue layer and placed flat on the body tissue layer at the interior of the body tissue layer;
said one limb comprising a channel for partially receiving the tube, a radius of curvature of said channel corresponding to an outer radius of the tube;
said other limb having a flat arc-shaped cross-section, a radius of curvature of the cross-section at the lower side of said one limb facing away from said one limb corresponding to an outer radius of the tube, and said other limb comprising a contour at its upper side facing towards said one limb, which avoids the body tissue layer from slipping-off from said other limb; and
the fixing mechanism comprising a hook protruding from said one limb, and an elastically extendible strap retainer having an end which is anchored to said one limb of the retaining element and a remainder which can be slung about the tube in said channel and then be hooked on said hook.
